(19) United States
(12) Patent Application Publication
(10) Pub. No.: US 2025/0222022 A1
(43) Pub. Date: Jul. 10, 2025

(54) INORGANIC COMPOUND WITH RATIO OF MAGNESIUM CHLORIDE TO MAGNESIUM SULFATE OF 1:0.64

(71) Applicant: Tomomi Toriyama, Shizuoka (JP)

(72) Inventor: Tomomi Toriyama, Shizuoka (JP)

(21) Appl. No.: 18/408,151

(22) Filed: Jan. 9, 2024

(51) Int. Cl. A61K 33/06; A61K 9/48; C01F 5/30; C01F 5/40

(52) U.S. Cl. CPC A61K 33/06; A61K 9/4833; C01F 5/30; C01F 5/40

(57) ABSTRACT

Magnesium chloride and magnesium sulfate are tightly bound together into one inorganic compound in water. In the present invention, the substance is named “siblesin” (registered trademark). The substance is not a mere mixture but is a substance having functions beyond human comprehension. As a result, the substance is highly effective against viral colds and seasonal infections caused by influenza virus, coronavirus, rotavirus, and so on. In particular, the substance is a specific medicine for ALS, which is an intractable disease.

### FIELD

The present invention relates to an inorganic compound that is used in an agent for medical use, health food, and so on. The inorganic compound has a ratio of magnesium chloride to magnesium sulfate of 1.0:0.64, which is equal to that of the two magnesium salts in sea water. As the unique properties of inorganic compounds, unlike organic compounds, inorganic compounds in which the ratio is 0.2 to 0.64 or 0.64 to 0.9 are also included in the present invention. A method for manufacturing an agent for medical use is also provided.

### BACKGROUND

Since 2020, there have been no cases of coronavirus infection on Awajishima, Shodoshima and Sadogashima, surrounded by the Japanese sea.

According to the WHO data in February 2020, there are no cases of virus infection in the island nations surrounded by the Oceania sea.

The reason for this was thought to be that magnesium salts in sea water equivalent to the substance of the present invention killed the virus.

In investigating this, the substance of the present invention was not noticed only by randomly mixing magnesium salts. Further, there was no prior art literature found that showed magnesium salts in sea water equivalent to the substance of the present invention had superior effect as a medical agent.

### SUMMARY

In the present teachings, an inorganic compound of magnesium with the same ratio as that in sea water was manufactured, and a therapeutic trial using the compound was performed.

### DETAILED DESCRIPTION

In the therapeutic trial of the present invention, a compound was manufactured using commercially available magnesium salts and was used as a sample. In 1,000 mL of water, 7.0 g of magnesium chloride hexahydrate and 2.1 g of anhydrous magnesium sulfate were dissolved, and a compound with the same concentration as that in sea water was thus manufactured and used. In this case, the ratio of magnesium chloride to magnesium sulfate is 1.0:0.64, which is the same concentration as that in sea water.

The subject was the inventor himself, who accidentally suffered from “achlorhydria (no production of gastric acid)” due to overtake of a gastrointestinal drug, H₂ blocker (Gaster 20), felt a dull pain in the back, had a CEA value of 9.6, and had symptoms of a viral cold with phlegm in the throat and inflammation in the mesentery. Since recovery by taking a cold medicine and dilute hydrochloric acid was insufficient, a solution of the inorganic compound of magnesium was taken.

The solution was taken 3 times a day between meals, 30 mL each time, for 20 days.

As a result, the dull pain in the back disappeared, the slight fever went down, the cold symptoms such as cough and phlegm completely disappeared. Furthermore, the colon movement was improved, constipation was resolved, and gastroptosis became normal. Moreover, hearing with a hearing aid was improved, and dry eyes returned to normal.

From the above, functions of healing the auditory nerves and the optic nerves were observed. Furthermore, it was inferred that the compound has functions of healing the gustatory nerves and the olfactory nerves.

Separately, the amount of the above aqueous solution was increased, and 60 mL thereof was taken. Then, during walking with flat feet due to aging, suddenly, walking stride became that of young people, and therefore, it was inferred that, surprisingly, the solution has the action of relaxing the nerves and muscles.

Accordingly, the compound is thought to be effective for a wide range of diseases, from viral colds and infectious diseases caused by influenza virus, coronavirus, and so on to intractable diseases such as Parkinson's disease and ALS, and it is believed that the compound must be a panacea due to the effect of an inorganic compound of magnesium present in sea water.

In addition, previously, a CT scan examination of the lungs showed frequent occurrence of cysts and alveolar destruction and disappearance due to long-term smoking. However, a recent X-ray examination showed that the whole lungs turned black and became normal. Accordingly, it was inferred that the compound is also effective for severe diseases such as pneumonia and lung cancer.

In order to manufacture the inorganic compound of magnesium of the present invention equivalent to the substance in sea water and make it as a medicine, commercially available raw materials can be used. For both (1) and (2), the ratio of MgCl₂ to MgSO₄ is 1.0:0.64.
(1) An aqueous solution of 210.0 mg of magnesium chloride hexahydrate and 62.5 mg of anhydrous magnesium sulfate, 272.5 mg in total, is gelled appropriately and is made into an oval soft capsule of 450 mg. One capsule corresponds to 15 mL of the above test solution.
(2) An aqueous solution of 210.0 mg of magnesium chloride hexahydrate and 128.0 mg of magnesium sulfate heptahydrate, 338.0 mg in total, is gelled appropriately and is made into an oval soft capsule of 450 mg. One capsule corresponds to 15 mL of the above test solution. Caution: The substance of the present invention loses its potency in capsule or powder form, so always use in aqueous solution formulation.

A specific manufacturing method is shown later.

Dosing method: In general, for both (1) and (2), 2 capsules are administered at a time, 3 times a day between meals, and the administration is continued for a predetermined period. For children, one-fourth to one-sixth the dose for an adult is administered once a day.

The substance of the present invention can also be applied as a fungicide for wheat and rice, fruit trees, and gardening (i.e., as an agricultural chemical), for livestock health and medical use, and as drinking water with a low concentration.

Method for manufacturing stock solution for “siblesin” (registered trademark) capsule

Example of manufacturing stock solution for oval soft capsule with net content of 0.45 g
(1) MgCl₂ hexahydrate: 210.0 g
   MgSO₄: 62.5 g
(2) CaSO₄: 1.2 g
(3) KCl: 0.6 g
   Powdered agar: 5.0 g
   Water: 620.7 g
Total: 900.0 g
(1) It is preferable if magnesium chloride hexahydrate and anhydrous magnesium sulfate are mixed with each other in powder form;
(2) CaSO₄ is not easily dissolved and is therefore added at the end; and
(3) An aqueous solution of KCl dissolves powdered agar well; therefore, it is sufficiently dissolved by stirring at about 80° C. Subsequently, a white to slightly yellow gel forms at room temperature.

When 900 g of the gel is manufactured, 2,000 oval soft capsules with a net content of 0.45 g can be manufactured according to calculations.

1. An inorganic compound of magnesium chloride and magnesium sulfate, bound together into one substance, for manufacturing an agent for medical use, wherein
a weight ratio of magnesium chloride to magnesium sulfate is 1.0:0.64, and
inorganic compounds in which the ratio is 0.2 to 0.64 or 0.64 to 0.9 are also included in the one substance.

2. A method for manufacturing a solution containing an inorganic compound of magnesium for manufacturing an agent, the method comprising:
dissolving raw materials in water at a weight ratio of magnesium chloride to magnesium sulfate of 1.0:0.64; and
filling a soft capsule or the like with the resulting solution.
